(19) United States
(12) Patent Application Publication
(10) Pub. No.: US 2011/0256629 A1
(43) Pub. Date: Oct. 20, 2011

(54) FULLY AUTOMATIC BIOCHEMICAL ANALYZER AND ANALYZING METHOD THEREOF

(75) Inventors: Zhihong Wang, Shenzhen (CN); Chuanfen Xie, Shenzhen (CN)

(73) Assignee: SHENZHEN MINDRAY BIO-MEDICAL ELECTRONICS CO., LTD., Shenzhen (CN)

(21) Appl. No.: 13/085,304

(22) Filed: Apr. 12, 2011

(30) Foreign Application Priority Data
Apr. 14, 2010 (CN) 201010153069.X

(51) Int. Cl. G01N 35/00; G01N 33/48

(52) U.S. Cl. 436/45; 422/64

(57) ABSTRACT

A fully automatic biochemical analyzer and an analyzing method thereof are provided, in which the fully automatic biochemical analyzer includes two concentric reagent trays and two driving systems. The two driving systems respectively drive the two reagent trays to rotate, so the two reagent trays are capable of independent rotation, thereby avoiding various drawbacks resulting from required simultaneous rotation and stopping of the two reagent trays. The two reagent trays are concentrically disposed. Under a particular limitation of the table size, more reagent positions can be disposed, thereby increasing the number of items that can be analyzed at the same time by the analyzer.

### CROSS-REFERENCE TO RELATED APPLICATIONS

This application claims the benefit of Chinese Patent Application No. 201010153069.X, filed on Apr. 14, 2010, which is incorporated herein by reference in its entirety.

### TECHNICAL FIELD

The following disclosure relates to biochemical analyzers.

### BRIEF DESCRIPTION OF THE DRAWINGS

FIG. 1 is a schematic structural view of a fully automatic biochemical analyzer;

FIG. 2 is a top view of a reagent tray;

FIG. 3 is a top view of a sample tray;

FIG. 4 is a schematic view of working positions of a reaction tray; and

FIGS. 5 and 6 are flow charts of methods for analyzing a reaction.

### DETAILED DESCRIPTION

Conventionally, a fully automatic biochemical analyzer includes a reagent tray, a sample tray, and a reaction tray, in which the sample tray is used for placing sample containers containing a sample to be tested, the reagent tray is used for placing reagent containers containing a reaction reagent for testing, and the reaction tray is used for placing reaction cups. By controlling rotation of the reagent tray, the sample tray, and the reaction tray in a coordinated manner, the sample and the reagent are respectively injected into the reaction cups. After the sample is mixed and reacted with the reagent, testing and analysis are performed. The number of reagent positions determines the number of items that can be analyzed at the same time by the analyzer, and the number of sample positions on the sample tray determines the number of samples that can be loaded at the same time in a continuous test.

The present disclosure is directed to a fully automatic biochemical analyzer that increases the usable reagent positions of the analyzer. According to one aspect, the present disclosure provides a fully automatic biochemical analyzer, including a first reagent tray for carrying reagent containers; a second reagent tray for carrying reagent containers, in which the second reagent tray is concentric with the first reagent tray; a first driving system for driving the first reagent tray to rotate; and a second driving system for driving the second reagent tray to rotate.

According to another aspect, a fully automatic biochemical analyzer includes: a reaction tray for carrying reaction cups; a first reagent tray for carrying reagent containers containing a first reagent; a second reagent tray for carrying reagent containers containing a second reagent, in which the second reagent tray is concentric with the first reagent tray; a first driving system for driving the first reagent tray to rotate; a second driving system for driving the second reagent tray to rotate; a first reagent dispensing mechanism for sucking reagent on the first reagent tray rotated to a reagent sucking position and injecting the reagent into the reaction cups; a second reagent dispensing mechanism for sucking reagent on the second reagent tray rotated to the reagent sucking position and injecting the reagent into the reaction cups; a sample tray for carrying sample containers; a sample dispensing mechanism for sucking sample on the sample tray rotated to a sample sucking position and injecting the sample into the reaction cups; a first stirring mechanism for performing a stirring operation for the reaction cup on a first stirring position; and a second stirring mechanism for performing a stirring operation for the reaction cup on a second stirring position.

According to yet another aspect, an analyzing method includes the following steps. The reaction tray is driven to rotate so that an empty reaction cup stops at a first reagent injecting position and a sample injecting position in sequence, or the reaction tray is driven to rotate so that the empty reaction cup stops at a sample injecting position and a first reagent injecting position in sequence. The first reagent dispensing mechanism injects the first reagent into the reaction cup stopped at the first reagent injecting position, and the sample dispensing mechanism injects the sample into the reaction cup stopped at the sample injecting position. The reaction tray is driven to rotate so that the reaction cup stops at the first stirring position of the reaction tray, and the first stirring mechanism performs the stirring for the reaction cup. The reaction tray is driven to rotate so that the reaction cup stops at the second reagent injecting position, and the second reagent dispensing mechanism injects the second reagent into the reaction cup stopped at the second reagent injecting position. Finally, the reaction tray is driven to rotate so that the reaction cup stops at the second stirring position of the reaction tray, and the second stirring mechanism performs the stirring of the second reagent in the reaction cup.

The present disclosure is further described in detail hereinafter with reference to specific embodiments and accompanying drawings. According to one embodiment, a fully automatic biochemical analyzer includes two concentric reagent trays in which more reagent positions can be disposed, thereby increasing the reagent positions of the biochemical analyzer. A first and/or third reagent can be placed on a first reagent tray, and a second and/or fourth reagent can be placed on the second reagent tray, thereby increasing the number of items that can be analyzed at the same time by the analyzer.

The two reagent trays may be concentrically disposed. An operating position on the reagent tray can be rotated to a position closer to an operator by controlling the rotation of the reagent tray, which is more convenient for the operator to pick and place reagent containers. Furthermore, compared with a method of disposing two reagent trays front and back, when the two reagent trays are concentrically disposed, more space can be saved and more reagent positions can be disposed.

In one embodiment, the fully automatic biochemical analyzer further includes two driving systems. The two driving systems respectively drive the two reagent trays to rotate, so the two reagent trays can rotate independently. Compared with an embodiment in which two reagent trays are driven by the same driving system, various drawbacks resulting from simultaneous rotation and stop required by the two reagent trays can be avoided.

The two reagent trays can be operated in parallel. Whether the first reagent tray is in a reagent sucking state does not influence the rotation of the second reagent tray. Similarly, whether the second reagent tray is in the reagent sucking state does not influence the rotation of the first reagent tray.

In order to conveniently control the rotation of the first reagent tray and the second reagent tray, the fully automatic biochemical analyzer may further include a first control key and a second control key. The first control key is used for controlling the first driving system to drive the first reagent tray to rotate a set angle, and the second control key is used for controlling the second driving system to drive the second reagent tray to rotate a set angle. The first control key and the second control key may be control buttons or control switches.

The fully automatic biochemical analyzer further includes a reagent dispensing mechanism. In an improved embodiment, the fully automatic biochemical analyzer includes two reagent dispensing mechanisms: a first reagent dispensing mechanism for injecting reagent on the first reagent tray into the reaction cups and a second reagent dispensing mechanism for injecting reagent on the second reagent tray into the reaction cups. As the two reagent trays can rotate independently, by disposing two reagent injecting positions on the reaction tray, the first reagent dispensing mechanism and the second reagent dispensing mechanism can act at the same time and respectively inject the reagent on the first reagent tray and the reagent on the second reagent tray into the reaction cups at the two reagent injecting positions, thereby increasing an analyzing efficiency.

In another improved embodiment, the two reagent trays of the fully automatic biochemical analyzer are concentrically disposed, in which the first reagent tray is located in the periphery of the second reagent tray, that is, the first reagent tray is an outer tray and the second reagent tray is an inner tray. Usually, the first reagent tray and the second reagent tray are respectively provided with a circle of reagent positions for placing the reagent cups at a circumference thereof. In order to increase the reagent positions, the reagent positions on the reagent tray are uniformly and closely distributed at the circumference of the reagent tray.

At least one fixed barcode scan window is left on the outer tray. In one embodiment, a single barcode scan window is used. The barcode scan window is a space between two adjacent reagent positions, in which the width of the space is at least larger than a width required for scanning the barcode of the reagent container on the second reagent tray. When the barcode scan window is in alignment with a barcode scanner, the barcode scanner can scan the barcode on the inner tray through the fixed barcode scan window disposed on the outer tray. After the barcode scan window is disposed on the outer tray, the fully automatic biochemical analyzer according to the embodiment only needs to use one barcode scanner to scan the barcodes on the outer tray and inner tray of the reagent tray.

In one embodiment, the reagent positions can be closely disposed on the outer tray of the reagent tray, thereby increasing the number of reagent positions on the reagent tray. When the barcode scanner needs to scan the barcode on the outer tray of the reagent tray, the first driving system drives the outer tray of the reagent tray to rotate so that the reagent containers on the outer tray of the reagent tray are scanned by the barcode scanner in sequence. When the barcode scanner needs to scan the barcode on the inner tray of the reagent tray, the first driving system drives the outer tray of the reagent tray to rotate so that the barcode scan window on the outer tray of the reagent tray stops at the scanning position of the barcode scanner. The second driving system drives the inner tray of the reagent tray to rotate so that the reagent containers on the inner tray of the reagent tray are scanned by the barcode scanner in sequence through the barcode scan window.

Similarly, in order to increase the sample positions of the fully automatic biochemical analyzer, the fully automatic biochemical analyzer includes a first sample tray for carrying sample containers, and a second sample tray for carrying sample containers, in which the first sample tray is concentric with the second sample tray. The fully automatic biochemical analyzer may further include a third driving system and a fourth driving system, in which the third driving system is used for driving the first sample tray to rotate and the fourth driving system is used for driving the second sample tray to rotate.

As a result of the design, the sample tray of the fully automatic biochemical analyzer is also a structure of two concentric trays, which can be driven independently, thereby not only increasing the sample positions of the fully automatic biochemical analyzer, but also making sample loading more convenient. When the first sample tray works, the operator can add samples on the second sample tray. Similarly, when the second sample tray works, the operator can add samples on the first sample tray.

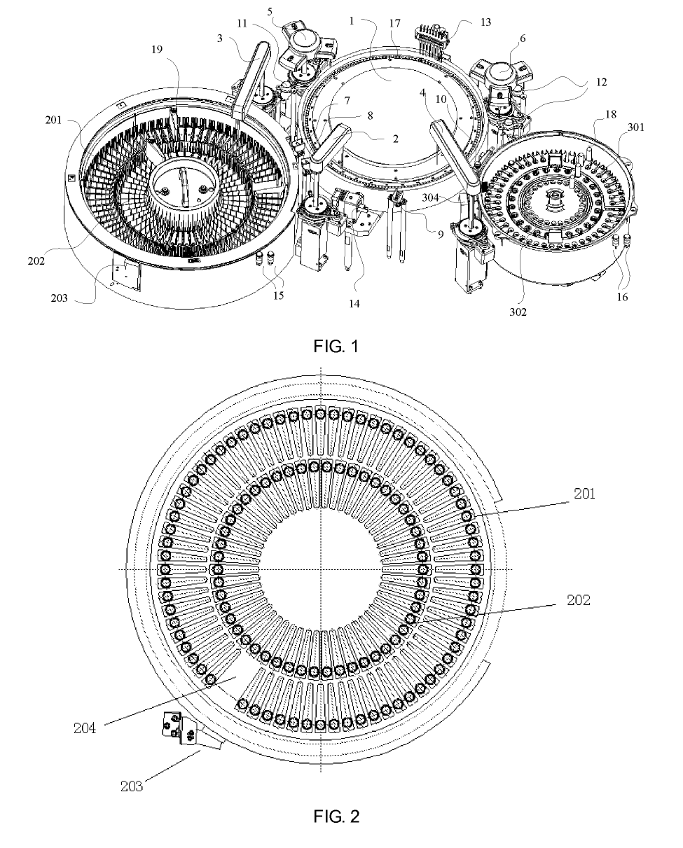

FIG. 1 shows a fully automatic biochemical analyzer. Referring to FIG. 1, the fully automatic biochemical analyzer may include a first reagent tray 201, a second reagent tray 202, an outer sample tray 301, an inner sample tray 302, a reaction tray 1, a first reagent dispensing mechanism 2, a second reagent dispensing mechanism 3, a sample dispensing mechanism 4, a first stirring mechanism 5, a second stirring mechanism 6, a first reagent dispensing mechanism cleaning pool 8, a second reagent dispensing mechanism cleaning pool 7, a sample dispensing mechanism cleaning pool 9, a sample dispensing mechanism enhanced cleaning position 10, two cleaning pools 11 of the first stirring mechanism, two cleaning pools 12 of the second stirring mechanism, a reaction cup automatic cleaning mechanism 13, and a photoelectric measuring module 14. Furthermore, the fully automatic biochemical analyzer may include an optional reagent barcode scanner 203 and a sample barcode scanner 304. The first reagent tray 201 and the second reagent tray 202 may further have a refrigerating function and the inner circle of the inner sample tray 302 may also have a refrigerating function.

In one embodiment, reagent containers 19 can be placed on the reagent tray and all positions on the reagent tray may support the barcode scanning. Sample containers 18 can be placed on the sample tray, and the outer circle of the outer sample tray 301, the inner circle of the outer sample tray 301, and the outer circle of the inner sample tray 302, all of which may support the barcode scanning. The right side outside the first reagent tray 201 may be provided with two reagent tray rotation control buttons 15. The right side of the outer sample tray 301 may be provided with two sample tray rotation control buttons 16, in which the two buttons may have indicator lamps so as to indicate the working state of the sample trays at the same time.

In one embodiment, the reaction tray 1 is arranged in the middle of the worktable of the instrument. Several (for example, 165) reaction cups 17 are arranged at an equal interval along the circumference of the reaction tray. The reaction cup is used as both a container for reaction of reaction liquids and a utensil for color comparison. The reaction tray has a constant temperature device so that the reaction liquids in the reaction cup can be maintained within a set temperature range. The outer circumference of the reaction tray is arranged with the reaction cup automatic cleaning mechanism 13, the first stirring mechanism 5, two cleaning pools 11 of the first stirring mechanism, the second reagent dispensing mechanism 3, the cleaning pool 7 of the second reagent dispensing mechanism, the first reagent tray 201, the second reagent tray 202, the first reagent dispensing mechanism 2, the cleaning pool 8 of the first reagent dispensing mechanism, the photoelectric measuring module 14, the sample dispensing mechanism 4, the cleaning pool 9 of the sample dispensing mechanism, the outer sample tray 301, the inner sample tray 302, the second stirring mechanism 6, and two cleaning pools 12 of the second stirring mechanism.

As shown in FIG. 2, the first reagent tray 201 and the second reagent tray 202 may be two concentric trays and arranged on the left side of the reaction tray on the worktable of the analyzer. The two trays are capable of independent rotation in one embodiment. The first reagent tray 201 is located on the outer circle and the second reagent tray 202 is located on the inner circle. At the circumference of the first reagent tray 201, a vacant position is arranged as the reagent barcode scan window 204. On the rest of the positions, several (for example, 70) reagent positions are distributed at an equal interval where the reagent containers 19 are correspondingly placed for carrying a first reagent and a third reagent used in a biochemical test, and an enhanced cleaning agent required by the first reagent dispensing mechanism.

At the circumference of the second reagent tray 202, several (for example, 50) reagent positions are distributed at an equal interval where the reagent containers 19 are correspondingly placed for carrying a second reagent and a fourth reagent used in the biochemical test, and an enhanced cleaning agent required by the second reagent dispensing mechanism. The first reagent tray 201 and the second reagent tray 202 share a reagent barcode scanner 203 which is disposed on the outer circumference of the first reagent tray 201. FIG. 2 is a schematic plan view of the first reagent tray 201 and the second reagent tray 202. As shown in FIG. 2, the first reagent tray 201 and the second reagent tray 202 have a reagent refrigerating function. The reagent refrigerating function is realized through a refrigeration module, in which the refrigeration module operates using a water cooling circulation method. In another embodiment, the reagent outer tray can be disposed with two or three reagent barcode scan windows.

The two rotation control buttons of the reagent tray are respectively used for controlling the rotation of the first reagent tray 201 and the second reagent tray 202 for the convenience of replacing the reagent. When the analyzer is in a non-test state, the button is pressed so the corresponding reagent tray rotates a set angle in a particular direction. The user can rotate the required reagent position to a position convenient for observation and operation.

The first reagent dispensing mechanism may be located at a front position between the first reagent tray 201 and the reaction tray and may include a reagent needle driving mechanism and a first reagent needle. A rotating trace of the first reagent needle respectively passes the reagent sucking position on the first reagent tray 201, the first/third reagent injecting position on the reaction tray, and the center of a first reagent needle cleaning pool between the first reagent tray 201 and the reaction tray.

The second reagent dispensing mechanism may be located at a rear position between the first reagent tray 201 and the reaction tray and may include a reagent needle driving mechanism and a second reagent needle. A rotating trace of the first reagent needle respectively passes the reagent sucking position on the second reagent tray 202, the second/fourth reagent injecting position on the reaction tray, and the center of a second reagent needle cleaning pool between the first reagent tray 201 and the reaction tray.

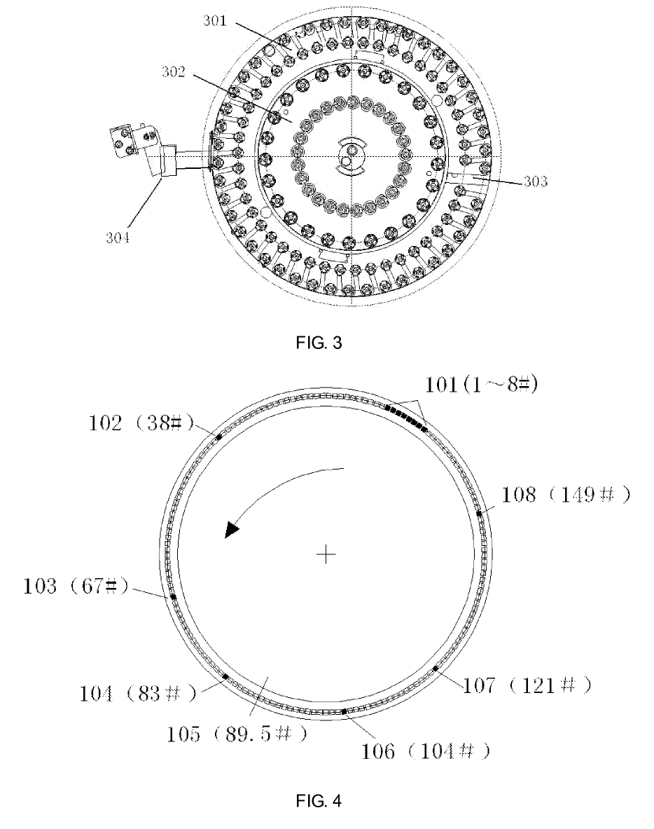

As shown in FIG. 3, the outer sample tray 301 and the inner sample tray may be two concentric trays and arranged at front positions on the right side of the reaction tray on the worktable of the analyzer. The two trays are capable of independent rotation. In one embodiment, the outer sample tray 301 is arranged with an inner circle and an outer circle of sample positions, in which the inner circle and the outer circle respectively reserve a sample barcode scan window 303 at the circumference, and at the rest of the positions, 45 sample positions are distributed at an equal interval. The inner sample tray 302 is arranged with an inner circle and an outer circle of sample positions, in which 25 positions are distributed at an equal interval respectively on the inner circle and the outer circle and all the sample positions are used for carrying sample containers 18. The 25 positions on the inner circle have a refrigerating function and can be used for placing quality control solution and calibration solution or used as normal sample positions.

Both the inner circle and the outer circle can be provided with a refrigerating function. The sample refrigerating function may be realized through a refrigeration module, in which the refrigeration module operates by using a water cooling circulation method. The outer circles of the outer sample tray 301 and the inner sample tray 302 share one barcode scanner. The sample barcode scanner 304 is installed at the outer circumference of the outer sample tray 301. FIG. 4 is a schematic plan view of the outer sample tray 301 and the inner sample tray 302.

Two rotation control buttons of the sample tray are respectively used for controlling the rotation of the outer sample tray 301 and the inner sample tray 302. Each of the control buttons has an indicator lamp to indicate the working states of the two sample trays at the same time. When the analyzer is in a non-test state, the button is pressed so the corresponding sample tray rotates a set angle in a particular direction. The user can rotate the required sample position to a position convenient for observation and operation. During the test, a sample needle can only sucks sample from one of the sample trays in one period.

In one embodiment, while performing a number of tests, the indicator lamp corresponding to the sample tray having samples being tested in the period is set to a ON state; the indicator lamp corresponding to the sample tray having samples to be tested after two periods may be set to a flickering state in the two periods; and the indicator lamp corresponding to the sample tray having samples to be tested after more than two periods or no sample to be tested may be set to an OFF state. Therefore, through different states of the indicator lamp on the button, the user can conveniently distinguish the working states of the two sample trays, so as to securely and rapidly add samples on the sample tray which does not rotate in a certain period during the test according to the requirements without interrupting the current test. Meanwhile, when adding samples during the test, the user can control the rotation of the tray through the button, so as to rotate the sample position to be operated to a position away from the rotating trace of the sample needle, thereby making the operation more secure.

In one embodiment, the sample dispensing mechanism is located at a front position between the reaction tray and the outer sample tray 301 and may include a sample needle driving mechanism and a sample needle. A rotating trace of the sample needle respectively passes the sample injecting position on the reaction tray, the diluted sample sucking position on the reaction tray, the sample sucking position on the outer circle of the outer sample tray 301, the sample sucking position on the inner circle of the outer sample tray 301, the sample sucking position on the outer circle of the inner sample tray 302, the sample sucking position on the inner circle of the inner sample tray 302, a sample needle enhanced cleaning position, and the center of a sample needle cleaning pool.

In one embodiment, the sample needle cleaning pool is located outside the reaction tray and on the rotating trace of the sample needle. The sample needle enhanced cleaning position is located between the outer circle of the sample tray and the reaction tray and on the rotating trace of the sample needle.

The photoelectric measuring module may be located at the front side of the reaction tray on the worktable and used for measuring the photoelectric data of the solution in the reaction cups.

The first stirring mechanism may be located at the left rear side of the reaction tray on the worktable, and may used for stirring after the sample is injected and stirring and mixing after the third reagent is injected. The second stirring mechanism may be located at the right side of the reaction tray on the worktable, and may be used for stirring after the second reagent is injected and stirring and mixing after the fourth reagent is injected. The first stirring mechanism and the second stirring mechanism are respectively disposed with three stirring rods at an equal interval around the rotating circumference. Through the rotation and vertical movement of the first stirring mechanism, the three stirring rods on the first stirring mechanism respectively arrive at the first stirring position on the reaction tray and the two cleaning pools of the first stirring mechanism. Similarly, through the rotation and vertical movement of the second stirring mechanism, the three stirring rods on the second stirring mechanism respectively arrive at the second stirring position on the reaction tray and the two cleaning pools of the second stirring mechanism.

The four cleaning pools of the two stirring mechanisms may be respectively located at the rotating traces of the stirring rods on each stirring mechanism outside the reaction tray.

The reaction cup automatic cleaning mechanism may be located at the right rear side of the reaction tray on the worktable and may be used for realizing the automatic cleaning of the reaction cups for the test.

The rotation and position of the reaction tray, the first reagent tray, the second reagent tray, the inner sample tray, and the outer sample may be realized respectively through a stepping motor, a transmission device, and a control device. The vertical movement, the horizontal rotation and the positioning of movement of the first reagent dispensing mechanism, the second reagent dispensing mechanism, the sample dispensing mechanism, the first stirring mechanism, and the second stirring mechanism can be realized respectively through the stepping motor, the transmission device, and the control device. The vertical movement and the positioning of the reaction cup automatic cleaning mechanism can be realized through the stepping motor, the transmission device, and the control device.

FIG. 4 is a schematic view of working positions of the reaction tray according to the layout described above. The 165 positions on the reaction tray may be sequentially numbered in the counterclockwise direction to form a position coordinate system of the reaction tray. 101 is the reaction cup automatic cleaning position and the corresponding position coordinate number thereof on the reaction tray is #1 to #8. 102 is the first stirring position on the reaction tray and the corresponding position coordinate number thereof on the reaction tray is #38. 103 is the second/fourth reagent injecting position on the reaction tray, and the corresponding position coordinate number thereof on the reaction tray is #67. 104 is the first/third reagent injecting position on the reaction tray and the corresponding position coordinate number thereof on the reaction tray is #83. 105 is the photoelectric measurement position on the reaction tray and the corresponding position coordinate number thereof on the reaction tray is #89.5. 106 is the diluted sample sucking position on the reaction tray, and the corresponding position coordinate number thereof on the reaction tray is #104. 107 is the sample injecting position on the reaction tray, and the corresponding position coordinate number thereof on the reaction tray is #121. 108 is the second stirring position on the reaction tray and the corresponding position coordinate number thereof on the reaction tray is #149.

The structural layout of the each part in the fully automatic biochemical analyzer in the above description can also be adjusted according to the specific design. For example, the reagent trays may be disposed on the right side of the worktable, the sample trays may be disposed on the left side of the worktable, and the positions of the reagent needle, the sample needle, the stirring structures, and the cleaning structures may be correspondingly adjusted.

In another embodiment, the two reagent trays, apart from being concentric inner and outer trays, may further be concentric upper and lower trays.

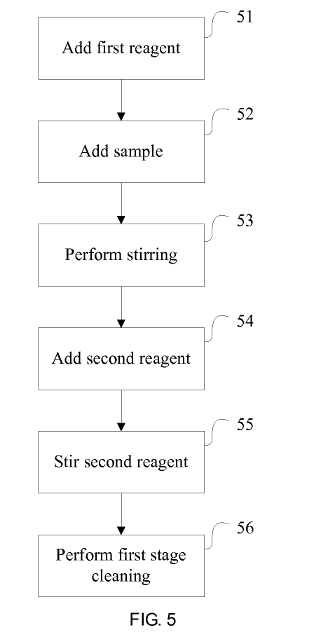

According to the structure of the fully automatic biochemical analyzer in the above embodiment, taking one reaction cup as an example, as shown in FIG. 5, the analyzing method may include the following steps.

In Step 51, the reaction tray is driven to rotate so that an empty reaction cup stops at the first reagent injecting position, and the first reagent dispensing mechanism injects the first reagent into the reaction cup stopped at the first reagent injecting position.

In Step 52, the reaction tray is driven to rotate so that the reaction cup stops at the sample injecting position, and the sample dispensing mechanism injects the sample into the reaction cup stopped at the sample injecting position.

In the above two steps, the reaction cup can be made to first stop at the sample injecting position so the sample dispensing mechanism injects the sample into the reaction cup stopped at the sample injecting position, and then stop at the first reagent injecting position so the first reagent dispensing mechanism injects the first reagent into the reaction cup stopped at the first reagent injecting position.

In Step 53, the reaction tray is driven to rotate so that the reaction cup stops at the first stirring position of the reaction tray, and the first stirring mechanism performs stirring for the reaction cup, that is, mixes the first reagent and the sample in the reaction cup.

In Step 54, the reaction tray is driven to rotate so that the reaction cup stops at the second reagent injecting position, and the second reagent dispensing mechanism injects the second reagent into the reaction cup stopped at the second reagent injecting position.

In Step 55, the reaction tray is driven to rotate so that the reaction cup stops at the second stirring position of the reaction tray, and the second stirring mechanism performs stirring of the second reagent in the reaction cup, that is, mixing the second reagent and the mixed liquid of the first reagent and the sample in the reaction cup. At this point, the test of the double reagent items in the reaction cup is ended.

In Step 56, the reaction tray is driven to rotate so that the reaction cup stops at the first stage of the reaction cup automatic cleaning position, and the reaction cup automatic cleaning position performs the first stage cleaning of the reaction cup.

In the above steps, after the first reagent or the sample is initially injected into the empty reaction cup, the first reagent or the sample can also be stirred.

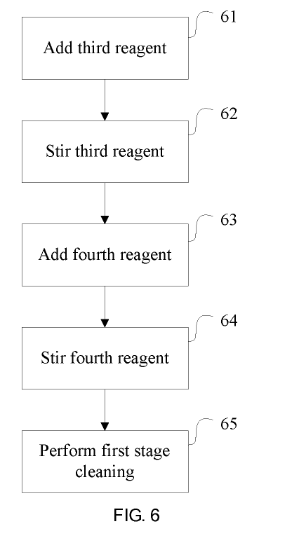

When three reagent items exist in the reaction cup test, the reagent cup containing the third reagent is placed on the first reagent tray. At this time, after the stirring of the second reagent, the reaction cup does not need cleaning. As shown in FIG. 6, the following steps may be performed.

In Step 61, the reaction tray is driven to rotate so that the reaction cup stops at the third reagent injecting position, the third reagent injecting position is the same as the first reagent injecting position in Step 51, and the first reagent dispensing mechanism injects the third reagent into the reaction cup stopped at the third reagent injecting position.

In Step 62, the reaction tray is driven to rotate so that the reaction cup stops at the first stirring position of the reaction tray, and the first stirring mechanism performs stirring of the third reagent in the reaction cup, that is, mixing the third reagent and the reaction liquid in the reaction cup.

When four reagent items exist in the reaction cup test, the reagent cup containing the fourth reagent is placed on the second reagent tray. After the first stirring mechanism performs the stirring of the third reagent in the reaction cup, the following steps may be performed.

In Step 63, the reaction tray is driven to rotate so that the reaction cup stops at the fourth reagent injecting position, the fourth reagent injecting position is the same as the second reagent injecting position, and the second reagent dispensing mechanism injects the fourth reagent into the reaction cup stopped at the fourth reagent injecting position.

In Step 64, the reaction tray is driven to rotate so that the reaction cup stops at the second stirring position of the reaction tray, and the second stirring mechanism performs stirring of the fourth reagent in the reaction cup. At this point, the test of the four reagent items in the reaction cup is ended.

In Step 65, the reaction tray is driven to rotate so that the reaction cup stops at the first stage of the reaction cup automatic cleaning position, and the reaction cup automatic cleaning position performs the first stage cleaning of the reaction cups.

When the sample needs to be diluted, and before the sample dispensing mechanism injects the sample into the reaction cups, the following steps of diluting the sample may be performed.

1. The reaction tray is driven to rotate so that the reaction cup for containing the diluted sample stops at the first reagent injecting position, and the first reagent dispensing mechanism injects distilled water into the reaction cup stopped at the first reagent injecting position.

2. The reaction tray is driven to rotate so that the reaction cup for containing the diluted sample stops at the sample injecting position, and the sample dispensing mechanism injects the sample to be diluted into the reaction cup stopped at the sample injecting position.

3. The reaction tray is driven to rotate so that the reaction cup for containing the diluted sample stops at the first stirring position of the reaction tray, and the first stirring mechanism performs the stirring of the sample to be diluted in the reaction cup and completes the dilution of the sample.

4. When the diluted sample needs to be injected into the reaction cup to be tested, the reaction tray is driven to rotate so that the reaction cup to be tested stops at the sample injecting position and the reaction cup for containing the diluted sample stops at a diluted sample sucking position, and the sample dispensing mechanism sucks a certain amount of the diluted sample from the reaction cup stopped at the diluted sample sucking position and injects the diluted sample into the reaction cup stopped at the sample injecting position.

The above test process is divided into several continuous working periods, in which the duration of each working period is equal. In each working period, all action components work according to a specified action sequence and complete actions of the sample adding, the first/second/third/fourth reagent adding, stirring, the photoelectric data collection, and automatic cleaning of the reaction cup. The working periods are performed continuously and repeatedly in this way so that test of multiple items can be accomplished in a continuous and efficient manner.

In each working period, the reaction tray performs one stop and one rotation in sequence. When the reaction tray is stopped, the sample dispensing mechanism, the first reagent dispensing mechanism, the second reagent dispensing mechanism, the first stirring mechanism, the second stirring mechanism, and the automatic cleaning mechanism respectively perform the sample injecting, the reagent injecting, the stirring and mixing, and the reaction cup cleaning according to the requirements of the test process. During the rotation of the reaction tray, the photoelectric data collection of the reaction cup passing the photoelectric measurement position on the reaction tray is performed.

Correspondingly, the outer sample tray or the inner sample tray stops the required sample containers at the sample sucking position in each period. The sample needle sucks the sample at the sample sucking position on the sample tray and injects the sample into the reaction cup at the sample injecting position on the reaction tray. After injecting the sample in each period, the cleaning of the inner and outer walls of the needle is performed on the sample needle at the sample needle cleaning pool.

Similarly, the first reagent tray stops the required reagent cup at the reagent sucking position in each period. The first reagent dispensing mechanism sucks the reagent at the reagent sucking position on the first reagent tray and injects the reagent at the first/third reagent injecting position on the reaction tray. After injecting the reagent in each period, the cleaning of the inner and outer walls of the needle is performed on the first reagent needle at the first reagent needle cleaning pool. The actions of the second reagent tray and the second reagent dispensing mechanism are similar to those of the first reagent tray and the first reagent dispensing mechanism, so the details are not repeated here.

In one embodiment, during the stopping period of the reaction tray, the three stirring rods of the stirring mechanism respectively perform the first stage cleaning, the second stage cleaning, and the reaction liquid stirring. During the rotation period of the reaction tray, the stirring mechanism rotates and changes the positions of the three stirring rods, so that the stirring rod performing the reaction liquid stirring in the last period stops at the first stage cleaning position, the stirring rod performing the first stage cleaning in the last period stops at the second stage cleaning position, and the stirring rod performing the second stage cleaning in the last period stops at the reaction liquid stirring position. The operation of the first stirring mechanism and the second stirring mechanism are similar.

As the reagent and the sample injected into the reaction cups need some time for incubation, several periods might exist between the adjacent steps. In one embodiment, it is set that m reaction cups are increased or reduced in the rotating direction after the reaction tray rotates a circle, and the relationship between the number of the reaction cup positions X and the number of periods N required by the reaction tray to rotate a circle, and the number of cup positions Y rotated by the reaction tray in each period is: X=N*Y±m. The test process of the reaction cup is described in detail hereinafter, taking an example that the number of the reaction cup positions disposed on the reaction tray is 165. The number of periods required by the reaction tray to rotate a circle is set according to the structure and layout of the fully automatic biochemical analyzer. In the embodiment, it is set that the reaction tray needs four periods to rotate a circle and the number of cup positions rotated by the reaction tray in each period is 41. The period of injecting the first reagent into the reaction cup is defined as a first period. In combination with the schematic view of working positions of the reaction tray in FIG. 4, the test process of any reaction cup is as follows.

In the first period, the first reagent needle of the first reagent dispensing mechanism injects the first reagent into a certain clean reaction cup stopped at the first/third reagent injecting position 104 of the reaction tray. Subsequently, the first reagent in the reaction cup enters an incubation period.

In a 14th period, the reaction cup stops at the sample injecting position 107 on the reaction tray, and the sample needle of the sample dispensing mechanism injects the sample into the reaction cup.

In a 16th period, the reaction cup stops at the first stirring position 102 on the reaction tray, the first stirring mechanism performs stirring of the sample in the reaction cup, and the reaction of the single reagent item starts.

In a 65th period, if two reagent items exist, the reaction cup stops at the second/fourth reagent injecting position 103 on the reaction tray, and the second reagent needle of the second reagent dispensing mechanism injects the second reagent into the reaction cup.

In a 67th period, the reaction cup stops at the second stirring position 108 on the reaction tray, the second stirring mechanism performs stirring of the second reagent in the reaction cup, and the reaction of two reagent items starts.

In a 130th period, if a single reagent item or double reagent items exist, the test of the reaction cup is ended.

In a 136th period, the reaction cup stops at the first stage of the reaction cup automatic cleaning position 101, and the reaction cup automatic cleaning mechanism performs the first stage cleaning of the reaction cup. If three or four reagent items exist, the cleaning is not performed.

In a 166th period, if three/four reagent items exist, the reaction cup stops at the first/third reagent injecting position 104 on the reaction tray, and the first reagent needle injects the third reagent into the reaction cup.

In a 181st period, the reaction cup stops at the first stirring position 102 on the reaction tray, and the first stirring mechanism performs stirring of the third reagent in the reaction cup.

In a 230th period, the reaction cup stops at the second/fourth reagent injecting position 103 on the reaction tray, and the reagent needle 2 injects the fourth reagent into the reaction cup.

In a 232nd period, the reaction cup stops at the second stirring position 108 on the reaction tray, and the second stirring mechanism performs stirring of the fourth reagent in the reaction cup.

In a 295th period, the test of the three/four reagent items in the reaction cup is ended.

In a 301st period, the reaction cup stops at the first stage of the reaction cup automatic cleaning position 101, and the reaction cup automatic cleaning mechanism performs the first stage cleaning of the reaction cup.

In one embodiment, when the sample needs to be diluted, the steps of diluting the sample are as follows.

The number of the reaction cup is defined as being incremental in the counterclockwise direction.

In an Nth period, the reaction cup numbered M stops at the first/third reagent injecting position 104 on the reaction tray, and the first reagent needle injects the distilled water into the reaction cup.

In an (N+13)th period, the reaction cup M stops at the sample injecting position 107 on the reaction tray, and the sample needle injects the sample to be diluted into the reaction cup M.

In an (N+15)th period, the reaction cup M stops at the first stirring position 102 on the reaction tray, and the first stirring mechanism performs stirring for the reaction cup M. At this point, the dilution of the sample is completed, and the steps of injecting the first reagent and the sample can be performed

In an (N+68)th period, the reaction cup (M+17) stops at the first/third reagent injecting position 104 on the reaction tray, and the first reagent needle injects the first reagent required by the diluting test into the reaction cup (M+17).

In an (N+81)th period, the reaction cup M stops at the diluted sample sucking position 106 on the reaction tray and the reaction cup (M+17) stops at the sample injecting position 107 on the reaction tray. The sample needle sucks a certain amount of diluted sample from the reaction cup M stopped at the diluted sample sucking position 106 on the reaction tray, and discharges the diluted sample into the reaction cup (M+17) stopped at the sample injecting position 107 on the reaction tray.

In an (N+83)th period, the reaction cup (M+17) stops at the first stirring position 102 on the reaction tray, and the first stirring mechanism performs the stirring for the reaction cup (M+17). If only a single reagent item exists, the reaction starts. If double/three/four reagent items exist, in the subsequent corresponding periods, the actions of injecting reagent into the reaction cup (M+17) and stirring for the reaction cup (M+17) are added in sequence, which are the same as the normal test processes. Therefore, details are not repeated here.

Though the invention has been disclosed above with reference to various preferred embodiments, the preferred embodiments are not intended to be limiting. Persons of ordinary skill in the art can make simple derivations and variations without departing from the scope of the invention, which shall be defined by the accompanying claims.

What is claimed is:

1. A fully automatic biochemical analyzer, comprising:
a first reagent tray configured for carrying reagent containers;
a second reagent tray configured for carrying reagent containers, wherein the second reagent tray is concentric with the first reagent tray;
a first driving system configured for driving the first reagent tray to rotate; and
a second driving system configured for driving the second reagent tray to rotate.

2. The fully automatic biochemical analyzer according to claim 1, further comprising:
a first reagent dispensing mechanism configured for injecting reagent on the first reagent tray into reaction cups; and
a second reagent dispensing mechanism configured for injecting reagent on the second reagent tray into the reaction cups.

3. The fully automatic biochemical analyzer according to claim 1, further comprising a reagent barcode scanner, wherein the first reagent tray is located in the periphery of the second reagent tray, the first reagent tray and the second reagent tray are respectively provided with a circle of reagent positions for placing reagent containers at a circumference thereof, at least one space between two adjacent reagent positions on the first reagent tray is larger than a width required for scanning the barcode of the reagent container on the second reagent tray, and when the barcode scanner scans the reagent barcode on the second reagent tray, the first driving system drives the first reagent tray to rotate to a position at which the space is in alignment with the barcode scanner.

4. The fully automatic biochemical analyzer according to claim 1, further comprising:
a first control key configured for controlling the first driving system to drive the first reagent tray to rotate a set angle; and
a second control key configured for controlling the second driving system to drive the second reagent tray to rotate a set angle.

5. The fully automatic biochemical analyzer according to claim 1, further comprising:
a first sample tray configured for carrying sample containers;
a second sample tray configured for carrying sample containers, wherein the second sample tray is concentric with the first sample tray;
a third driving system configured for driving the first sample tray to rotate; and
a fourth driving system configured for driving the second sample tray to rotate.

6. An analyzing method for a fully automatic biochemical analyzer, wherein the fully automatic biochemical analyzer comprises a reaction tray configured for carrying reaction cups, a first reagent tray configured for carrying reagent containers containing a first reagent, a second reagent tray configured for carrying reagent containers containing a second reagent, wherein the second reagent tray is concentric with the first reagent tray, a first driving system configured for driving the first reagent tray to rotate, a second driving system configured for driving the second reagent tray to rotate, a first reagent dispensing mechanism configured for sucking reagent on the first reagent tray rotated to a reagent sucking position and injecting the reagent into the reaction cup, a second reagent dispensing mechanism configured for sucking reagent on the second reagent tray rotated to the reagent sucking position and injecting the reagent into the reaction cup, a sample tray configured for carrying sample containers, a sample dispensing mechanism configured for sucking sample on the sample tray rotated to a sample sucking position and injecting the sample into the reaction cup, a first stirring mechanism configured for performing stirring for the reaction cup on a first stirring position; and a second stirring mechanism configured for performing stirring for the reaction cup on a second stirring position, the method comprising:
driving the reaction tray to rotate so that an empty reaction cup stops at a first reagent injecting position and a sample injecting position in sequence, or driving the reaction tray to rotate so that the empty reaction cup stops at the sample injecting position and the first reagent injecting position in sequence, wherein the first reagent dispensing mechanism injects the first reagent into the reaction cup stopped at the first reagent injecting position, and the sample dispensing mechanism injects the sample into the reaction cup stopped at the sample injecting position;
driving the reaction tray to rotate so that the reaction cup stops at the first stirring position of the reaction tray, wherein the first stirring mechanism performs stirring for the reaction cup;
driving the reaction cup to rotate so that the reaction cup stops at the second reagent injecting position, wherein the second reagent dispensing mechanism injects the second reagent into the reaction cup stopped at the second reagent injecting position; and
driving the reaction cup to rotate so that the reaction cup stops at the second stirring position of the reaction tray, wherein the second stirring mechanism performs stirring of the second reagent in the reaction cup.

7. The analyzing method according to claim 6, wherein when test items are three reagent items, the first reagent tray is further used for carrying reagent containers containing a third reagent, and after the second stirring mechanism performs the stirring of the second reagent in the reaction cup, the method further comprises:
driving the reaction cup to rotate so that the reaction cup stops at the first reagent injecting position, wherein the first reagent dispensing mechanism injects the third reagent into the reaction cup stopped at the first reagent injecting position; and
driving the reaction cup to rotate so that the reaction cup stops at the first stirring position of the reaction tray, wherein the first stirring mechanism performs stirring of the third reagent in the reaction cup.

8. The analyzing method according to claim 7, wherein when the test items in the reaction cup are four reagent items, the second reagent tray is further used for carrying reagent containers containing a fourth reagent, and after the first stirring mechanism performs the stirring of the third reagent in the reaction cup, the method further comprises:
driving the reaction cup to rotate so that the reaction cup stops at the second reagent injecting position, wherein the second reagent dispensing mechanism injects the fourth reagent into the reaction cup stopped at the second reagent injecting position; and
driving the reaction cup to rotate so that the reaction cup stops at the second stirring position of the reaction tray, wherein the second stirring mechanism performs the stirring of the fourth reagent in the reaction cup.

9. The analyzing method according to claim 6, further comprising:
before the sample dispensing mechanism injects the sample into the reaction cup, diluting the sample;
driving the reaction tray to rotate so that the reaction cup for containing the diluted sample stops at the first reagent injecting position, wherein the first reagent dispensing mechanism injects distilled water into the reaction cup stopped at the first reagent injecting position;
driving the reaction tray to rotate so that the reaction cup for containing the diluted sample stops at the sample injecting position, wherein the sample dispensing mechanism injects the sample to be diluted into the reaction cup stopped at the sample injecting position;
driving the reaction tray to rotate so that the reaction cup for containing the diluted sample stops at the first stirring position of the reaction tray, wherein the first stirring mechanism performs stirring of the sample to be diluted in the reaction cup and completes the dilution of the sample; and
driving the reaction tray to rotate when the diluted sample is needed to be injected into the reaction cup to be tested so that the reaction cup to be tested stops at the sample injecting position and the reaction cup for containing the diluted sample stops at a diluted sample sucking position, wherein the sample dispensing mechanism sucks a certain amount of the diluted sample from the reaction cup stopped at the diluted sample sucking position and injects the diluted sample into the reaction cup stopped at the sample injecting position.

10. The analyzing method according to claim 6, wherein a relationship between a number of the reaction cup positions for placing the reaction cups disposed on the reaction tray and a number of periods required by the reaction tray to rotate a circle, an incremental number of cup positions in a rotation direction or an opposite direction thereof when the reaction tray rotates a circle, and a number of cup positions rotated by the reaction tray in each period, is: X=N*Y±m, where X is the number of the reaction cup positions on the reaction tray, N is the number of periods required by the reaction tray to rotate a circle, Y is the number of cup positions rotated by the reaction tray, m is the incremental number of cup positions in the rotation direction or the opposite direction thereof when the reaction tray rotates a circle, and one period means that the reaction tray performs one stop and one rotation.
